(19) United States
(12) Patent Application Publication
(10) Pub. No.: US 2005/0244353 A1
(43) Pub. Date: Nov. 3, 2005

(54) Method for achieving shape memory effects on hair by combining shape memory polymers with cationic active ingredients

(75) Inventors: Andreas Lendlein, Berlin (DE); Gunther Lang, Reinheim (DE); Karl Kratz, Berlin (DE); Thomas Krause, Darmstadt (DE); Axel Kalbfleisch, Darmstadt (DE); Jurgen Allwohn, Burgschwalbach (DE); Johannes Burghaus, Gross-Umstadt (DE); Gerhard Sendelbach, Darmstadt (DE); Angelika Beyer, Waldaschaff (DE); Detlef Mattinger, Bickenbach (DE); Stefen Uhl, Pfungstadt (DE); Susanne Birkel, Darmstadt (DE); Anja Duchscherer, Hadamar (DE); Tareq Ali Abdull Hasson, Aachen (DE); Hong-Yan Jiang, Aachen (DE); Simone Morsheim, Eschweiler (DE); Yasmin Ghazlan, Bremen (DE)

Correspondence Address: KALOW & SPRINGUT LLP, 488 MADISON AVENUE, 19TH FLOOR, NEW YORK, NY, 10022, US

(73) Assignee: MNEMOSCIENCE GMBH, Uebach-Palenberg (DE)

(21) Appl. No.: 10/510,889

(22) PCT Filed: Apr. 10, 2003
(86) PCT No.: PCT/EP03/03733

§ 371 Date: Jun. 30, 2005

(30) Foreign Application Priority Data
Apr. 10, 2002 (DE) 102 15 858.4
Jun. 24, 2002 (DE) 102 28 120.3

(51) Int. Cl. A61K 7/06

(52) U.S. Cl. 424/70.1

(57) ABSTRACT

Disclosed is a method for achieving a recallable hair deformation. A composition is applied onto hair, comprising at least one first active principle or active complex, which, alone or in combination with further compounds, is able, after application to the hair and after carrying out the treatment of hair in accordance with the present invention, to provide a shape memory effect. The composition comprises at least one second active principle, which is a cationic agent. The hair is brought into a defined (permanent) shape and the permanent shape is subsequently fixed by means of inducing a chemical or physical change of the applied agents, wherein after a desired or undesired deformation, the memory shape of the initial memory shape may be recovered by means of a physical stimulation. In addition to the permanent hairdo, a second (temporary) shape can be impressed. Furthermore, disclosed is a method for the recovery of a once programmed permanent hairdo, which has been brought into a temporary shape or which has been deformed in any other way.

The present invention concerns a method of providing a recallable hair shape transformation using a combination of cationic agents and active agents, which provide the hair with a shape memory effect, in particular shape memory polymers or macromers which may be cross-linked in order to provide shape memory polymers.

In the field of shaping of hair, a distinction is usually made between temporary hair-shaping methods and long-lasting, permanent hair-shaping methods. A temporary hair-shaping usually occurs in connection with the use of compositions on the basis of solutions or dispersions comprising hair-fixative polymers. Such products provide the hair, by means of the polymers used, with improved hold, volume, elasticity, body and shine. Such styling products facilitate, for example, in the form of a gel, the shaping and provision of a hairdo; they improve, in the form of hair spray, the condition of a prepared hairdo; and they increase, in the form of setting foams, the volume of the hair. A drawback in this connection is that the desired effects are of only relatively short duration, and that they are lost under external influences, such as combing, wind, high humidity, or contact with water. A permanent hair transformation usually occurs during a treatment of providing a permanent wave. During this treatment the disulfide bonds in the hair are cleaved by use of a reductive reaction, the hair is brought into the new shape, and this new shape is fixed by providing new disulfide bonds by means of an oxidative process.

A drawback in this respect, however, is that the required chemical treatment of the hair employing reduction agents and oxidation agents involves an impairment of the structure of the hair. A further drawback of the methods for hair shaping known up to now is that it is not possible to revert a once-performed transformation (shaping) using relatively simple means, i.e., it is not possible to change from one shape of a hairdo to another shape of a hairdo without troublesome processes.

JP 04-41416 discloses compositions in the field of cosmetic preparations for hair which comprise specific linear polyurethanes having a glass transition temperature T_{g} of from 40 to 90° C. The process for hair treatment disclosed therein corresponds to the treatment using conventional thermoplastic materials. After the administration of the composition, a hairdo is prepared at a temperature above T_{g}, and this hairdo is then fixed by means of cooling under T_{g}. During a subsequent rewarming to a temperature above T_{g}, the polymer softens and a new hairdo can be prepared. A method for a recallable, reversible hair-shaping (hair transformation), however, is not disclosed.

The object underlying the present invention was to provide compositions having an improved efficiency and performance for providing a recallable hair transformation (hair-shaping) enabling a high degree of recovery of a once-programmed hairdo. An improved efficiency or performance may for example be given by means of an improved adhesion on hair, an improved degree of recovery etc. A further object was to provide a method enabling a permanent hair-shaping without requiring an impairing intervention into the hair structure. A further object was the provision of a method which enables the reversion of a temporary transformation/shaping with a high accuracy in order to revert to a previously prepared, programmed permanent hairdo. A further object was also the provision of a method which enables in a simple manner to remedy with a high accuracy the deformation of a hairdo which has been introduced by external influences, in order to revert to a once prepared, programmed permanent hairdo.

This object has been solved by providing a method for hair treatment, comprising:
applying a hair treatment composition onto hair, wherein the hair treatment composition comprises at least a first active principle or an active complex, selected from or formed from compounds which, alone or in combination with further compounds, are suitable to provide a shape memory effect to hair after application onto the hair and after having carried out the treatment described in the following, and wherein the hair treatment composition comprises further a second active principle selected from cationic agents;
previously, at the same time or subsequently to the application of the hair treatment composition, shaping the hair in order to provide a defined (permanent) shape;
fixing this permanent shape by means of a chemical or physical change (transformation) of the applied active principles;
wherein after a desired or undesired deformation of the permanent shape the initial permanent shape can substantially be reconstituted by means of a physical stimulus.

One embodiment is concerned with a method for hair treatment using a composition comprising at least two compounds, which alone do show only minute or no shape memory properties but which, when applied in combination according to the method of the present invention provide the hair with a synergistically enhanced shape memory effect. In this respect it may be mentioned that the at least two compounds comprise either the first active principle mentioned above and the cationic agent disclosed above, or the two compounds form the active complex mentioned above.

A preferred embodiment concerns a method of treating hair, comprising:
applying the hair treatment composition onto hair, wherein the first active principle is a macromer, wherein the macromer
   a. comprises crosslinkable areas (segments) which are crosslinkable by means of chemical bonds and
   b. thermoplastic areas (segments) which are not chemically crosslinkable,
bringing the hair, previously, at the same time or subsequently, into a desired (permanent) shape and
fixing the shape by means of chemical crosslinking of the macromer, thereby providing the shape memory polymer,
characterized in that the shape memory polymer possesses at least one transition temperature Tₜᵣₐₙₛ.

A further object of the present invention is a method for impressing a second hairdo over a programmed, recallable first hairdo. In this method, a hairdo, programmed by means of the above-explained method (permanent shape) is heated to a temperature above Tₜᵣₐₙₛ. Subsequently, the hair is brought into the second (temporary) shape and the second shape is fixed by means of cooling to a temperature below Tₜᵣₐₙₛ.

A further subject of the present invention is a method for the recovery of a first hairdo, programmed by means of the above-explained method (permanent shape). Therein, a hairdo in a temporary shape or in the shape of a hairdo obtained by means of cold forming, is heated to a temperature above Tₜᵣₐₙₛ.

Shape-memory polymers in accordance with the present invention are polymers from which materials can be prepared which possess the property that they can be impressed with a desired shape (permanent shape), to which these materials, after deformation or after impressing a second shape (temporary shape), can revert spontaneously and without external forces by means of simple warming or by means of another energetic stimulus. Deformation and retransformation (recovery) can be carried out over multiple cycles. The degree of attainability of the initial, permanent shape, is during a first relaxation cycle consisting of deformation and recovery, usually somewhat smaller than during the subsequent cycles, most probably due to the removal of existing textures, defects, etc., initially present. A very high degree of recovery will, however, be attained during the subsequent relaxation cycles. The degree of recovery during the first relaxation cycle is preferably at least 30%, in particular at least 50% and the degree of recovery during the subsequent relaxation cycles is preferably at least 60%, more preferably at least 80%. This degree may, however, be also 90% or more. The degree of recovery can be measured in accordance with usual curl retention measurements by means of the determination of the length of a treated hair strand or by means of known, suitable stress-strain experiments.

The shape memory effect on hair is the property the a specific hairdo (permanent memory shape) may be substatially recalled, after a deformation, spontaneously and without the application of any external forces by simple heating or some other energetic stimulus, i.e. to a degree during the first recovery cycle of at least 30%, preferably at least 50%, and to a degree, during subsequent cycles of recovery of at least 60%, preferably at least 80% or 90%.

Macromers or pre-polymers in accordance with the present invention which can be crosslinked in order to provide shape-memory polymers, are polymers or oligomers wherein the fixation of an impressed, permanent shape occurs by means of chemical bonds connecting specific polymer strands or oligomer strands. The crosslinkage, by means of chemical bonds, can be provided by means of ionic or covalent bonds. The crosslinking reaction may be any suitable chemical reaction, for example, a salt formation reaction, a condensation reaction, an addition reaction, a substitution reaction or a reaction initiated photochemically or by means of a radical. The crosslinking reaction can occur using suitable catalysts or initiators or the crosslinking reaction can occur without the use of a catalyst. The crosslinking reaction can be initiated by means of a suitable energy source, for example, electromagnetic radiation, ultrasound, heat or mechanical energy. A combination of two or more methods for initiation can be employed in order to increase the efficiency or the velocity of the crosslinking reaction.

Shape-memory polymers which can be used in accordance with the present invention possess at least one transition temperature Tₜᵣₐₙₛ. This transition temperature may be a melting temperature Tₘ or a glass transition temperature T_{g}. Above Tₜᵣₐₙₛ, the polymer has a lower modulus of elasticity than below Tₜᵣₐₙₛ. The ratio of the modulus of elasticity below Tₜᵣₐₙₛ to above Tₜᵣₐₙₛ is preferably at least 20. The transition temperature Tₜᵣₐₙₛ is preferably above room temperature (20° C.), preferably this transition temperature is at least 30° C., in particular preferred 40° C. The transition temperature Tₜᵣₐₙₛ is the temperature above which the spontaneous recovery of the permanent shape, starting from the deformed shape or the temporary shape, occurs.

The terms “hairdos” or “shape of hair” in accordance with the present invention are to be understood broadly and comprise in particular also the degree of waviness or the degree of evenness of hair. A programmed hairdo in accordance with the present invention is any group of hairs which show a specific shape due to shape-memory polymers which have been crosslinked and which fix a permanent shape. Recovery of a programmed hairdo in accordance with present invention means that the programmed hairdo is recovered after a deformation, to a degree of at least 60%, preferably to a degree of at least 80%, in relation to the shape which can be obtained after a first relaxation cycle. The degree of recovery can, for example, be determined by means of the determination of the length of a hair strand or hair curl.

Suitable macromers or pre-polymers, which can be chemically crosslinked in order to provide shape-memory polymers are macromonomers which can be polymerized or which can be crosslinked by means of single chemical bonds. The chemically crosslinkable polymers are designated as thermoset polymers in WO 99/42147. The macromers and thermoset polymers disclosed in WO 99/42147 are suitable for use in accordance with the present invention and these materials are incorporated here by reference. Soft, thermoplastic segments (switching segments) having a transition temperature Tₜᵣₐₙₛ are crosslinked by means of chemical, preferably covalent bonds. Required are switching segments and fixation points (network points). The fixation points fix the permanent form while the switching segments fix the temporary shape. The shape memory effect is based on the change of the elasticity at, above, or below Tₜᵣₐₙₛ or during the transition from above to below Tₜᵣₐₙₛ or from below to above Tₜᵣₐₙₛ. The ratio of the modulus of elasticity below Tₜᵣₐₙₛ to above Tₜᵣₐₙₛ is preferably at least 20. The higher this ratio is, the more expressed the shape-memory effect can be obtained. Four types of thermoset polymers having shape-memory properties can be designated:

Network polymers, penetrating network polymers, semi-interpenetrating networks and mixed penetrating networks. Network polymers can be formed by means of the covalent connection of macromonomers, i.e., of oligomers or polymers having reactive terminal groups, preferably ethylenically unsaturated terminal groups, terminal groups which can be reacted by means of radical reactions or terminal groups which can be reacted by means of photochemical reactions. The crosslinking reaction can, for example, be initiated by means of light-sensitive or temperature-sensitive initiators, by means of red-ox systems or combinations thereof, or the reaction can be initiated without the use of initiators, e.g., using UV light, heat or mechanical energy. Interpenetrating networks are formed at least two components which are each crosslinked but not with the other component. Mixed interpenetrating networks are formed using at least two components, wherein one component is crosslinked by means of chemical bonds and the second component is crosslinked by means of physical interactions. Semi-interpenetrating networks are formed using at least two components, of which one is chemically crosslinkable, while the other one is not chemically crosslinkable. Both components, however, cannot be separated from one another by means of physical methods.

In principle, all synthetic or natural oligomers or polymers having reactive terminal groups or side chain groups, which provide the crosslinked shape-memory polymer with a suitable transition temperature Ttrans and suitable moduli of elasticity above and below Ttrans are suitable, wherein the terminal groups or the side chain groups are already present initially or are provided by means of a subsequent derivatization. These materials allow a crosslinking reaction using the above-identified methods. Suitable macromers are, for example, macromers having the following formula
A1-(X)ₙ-A2 (I)
wherein A1 and A2 designate reactive, chemically crosslinkable groups and wherein
—(X)n- designates a divalent, thermoplastic polymer or oligomer segement. A1 and A2 are preferably acrylate or methacrylate groups. The segment —(X)n- preferably designates a polyester segment, an oligoester segment, a polyalkylene glycol segment, an oligoalkylene glycol segment, a polyalkylene carbonate segment or a oligoalkylene carbonate segment, wherein the alkylene groups are preferably ethylene groups or propylene groups. Suitable macromonomers for the formation of thermoset polymers having shape-memory properties are oligo-(ε-caprolactone) or poly(ε-caprolactone), oligolactide or polylactide, oligoalkylene glycol, olyalkyleneglycol, e.g., polyethylene glycol or their block copolymers, wherein the polymers or oligomers possess at least two ethylenically unsaturated groups, which can be polymerized by means of a radical reaction, e.g., acrylates or methacrylates, wherein these groups are provided at a terminal position or at any side chain position.

The polymer segments can be chosen among natural polymers, such as, for example, segments derived from proteins or polysaccharides. Suitable are also synthetic polymer segments. Suitable natural polymer segments are proteins such as zein, modified zein, casein, gelatine, gluten, serum albumin or collagen, as well as polysaccharides such as alginate, cellulose, dextrane, pullulane or polyhyaluronic acid, as well as chitin, poly(3-hydroxyalkanoate), especially poly(β-hydroxybutyrate), poly(3-hydroxyoctanoate) or poly(3-hydroxy fatty acids). Suitable are also derivatives of natural polymer segments such as alkylated, hydroxyalkylated, hydroxylated or oxidated modifications.

Synthetically modified natural polymers are, for example, cellulose derivatives such as alkylcelluloses, hydroxyalkylcelluloses, cellulose ethers, cellulose esters, nitrocellulose, chitosan or chitosan derivatives, which are, e.g., obtained by means of alkyl substitution or hydroxyalkyl substitution at the nitrogen and/or oxygen. Examples are methylcellulose, ethylcellulose, hydroxypropylcellulose, hydroxypropylmethylcellulose, hydroxybutylmethylcellulose, celluloseacetate, cellulosepropionate, celluloseacetatebutyrate, celluloseacetatephthalate, carboxymethylcellulose, cellulosetriacetate or cellulosesulfate sodium salt. These materials are all designated in the following as “celluloses.”

Suitable synthetic polymer blocks include polyphosphazenes, poly(vinylalcohols), polyamides, polyesteramides, poly(amino-acids), polyanhydrides, polycarbonates, poly(lactide-co-glycolide), polyacrylates, polyalkylenes, polyacrylamides, polyalkylenglycoles, polyalkylenoxides, polyalkylenterephthalates, polyorthoesters, polyvinylethers, polyvinylesters, polyvinylhalogenides, polyvinylpyrrolidones, polyesters, polylactide, polyglycolide, polysiloxanes, polyurethanes and copolymers thereof. Examples of suitable polyacrylate are poly(methylmethacrylate), poly(ethylmethacrylate), poly(butylmethacrylate), poly(isobutylmethacrylate), poly(hexylmethacrylate), poly(isodecylmethacrylate), poly-(laurylmethacrylate), poly(phenylmethacrylate), poly(methylacrylate), poly(isopropylacrylate), poly(isobutylacrylate) or poly(octadecylacrylate). Suitable synthetic, biologically degradable polymer segments are polyhydroxyacids such as polylactide, polyglycolide and and copolymers thereof, poly-(ethylenterephthalate); poly(hydroxybuturic acid); poly-(hydroxyvaleric acid); poly[lactide-co-(ε-caprolactone)]; poly[glycolide-co-(ε-caprolactone)]; polycarbonates, poly-(aminoacids); poly(hydroxyalkanoates); polyanhydrides; poly-orthoesters as well as mixtures and copolymers thereof. Examples of polymer segments which are less readily biologically degradable are poly(methacrylic acid), poly(acrylic acid), polyamides, polyethylene, polypropylene, polystyrene, polyvinylchloride, polyvinylphenol as well as mixtures and copolymers thereof.

In a preferred embodiment of the present invention, the composition comprises a mixture of (A) macromers which are substituted with at least two reactive, crosslinkable groups, and (B) macromers, which are substituted with only one reactive group. Suitable additional macromers are, for example, macromers of the following general formula:
R—(X′)ₙ-A3 (II)
wherein R designates a monovalent organic residue, A3 designates a reactive, chemically crosslinkable group and —(X′)ₙ designates a divalent, thermoplastic polymer segment or oligomer segment. A3 preferably represents an acrylate group or a methacrylate group. The segment —(X′)ₙ— preferably represents a polyalkyleneglycol, monoalkylethers thereof, or block copolymers thereof, wherein the alkylene groups preferably are ethylene or propylene groups and wherein the alkyl groups preferably comprise from 1 to 30 carbon atoms. Particularly preferred are mixtures of (A) polyalkyleneglycoles or polycaprolactones having terminal ester groups comprising acrylic acid or methacrylic acid and (B) polyalkyleneglycol-monoalkylethers, having one terminal ester group comprising acrylic acid or methacrylic acid wherein the alkylene groups are preferably ethylene or propylene groups and the alkyl groups are preferably C1-bis C30-alkyl groups.

As component (A) the following are, e.g., suitable: poly(ε-caprolactone)-dimethacrylate, poly(DL-lactide)-dimethacrylate, poly(L-lactide-co-glycolide)-dimethacrylate, poly(ethyleneglycol)dimethacrylate, poly(propyleneglycol)dimethacrylate, PEG-block-PPG-block-PEG-dimethacrylate, poly(ethyleneadipate)-dimethacrylate, hexamethylencarbonat-dimethacrylate.

Suitable as component (B) are, e.g., the following: poly(ethyleneglycol)monoacrylate, poly(propyleneglycol)monoacrylate and monoalkylethers thereof.

A further particular embodiment relates to a method of treating hair, comprising:
applying a composition onto hair, wherein the first active principle of the composition applied to hair is a shape memory polymer, which comprises at least two transition temperatures Tₜᵣₐₙₛ and T′ₜᵣₐₙₛ, and wherein this shape memory polymer comprises at least one hard segment with a first transition temperature T′ₜᵣₐₙₛ, which is crosslinkable by means of physical interactions, wherein the first transition temperature is above room temperature, preferably more than 10° C. above room temperature, and at least one soft segment having a second transition temperature Tₜᵣₐₙₛ, which lies below T′ₜᵣₐₙₛ, preferably at least 10° C. below T′ₜᵣₐₙₛ,
previously, at the same time or subsequently shaping the hair to a defined (permanent) shape and, subsequently, fixing the shape by means of a physical cross-linking of the shape memory polymer.

The shaping of hair occurs suitably under warming to a temperature of at least T′ₜᵣₐₙₛ and the shape of the hair is fixed by means of cooling to a temperature below T′ₜᵣₐₙₛ. Room temperature in this connection refers generally to ambient temperature, preferably at least 20° C. and, in warmer climates, preferably at least 25° C. The application of the composition onto the hair can be carried out in different ways, for example directly by spraying or in an indirect manner by applying the composition first to the hand or a suitable device, for example a comb, a brush etc, followed by a subsequent application within or onto the hair. The consistency of the composition may be the consistency of a solution, a dispersion, a lotion, a thickened lotion, a gel, a foam, a semi-solid composition, waxy or creamy.

A further embodiment of the invention is a method for impressing a second hairdo over a programmed, recallable first hairdo. During this method the hairdo (permanent shape), programmed by means of the above-mentioned method, is warmed to a temperature between T′ₜᵣₐₙₛ and Tₜᵣₐₙₛ. Subsequently the hair is brought into the second (temporary) shape and this second shape is fixed by means of cooling to a temperature below Tₜᵣₐₙₛ.

A further embodiment of the present invention is a method for recalling a programmed first hairdo (permanent shape), prepared by means of the above-mentioned method. In this method the hairdo in a temporary shape or in the shape of a hairdo being obtained by cold-forming, is warmed to a temperature above Tₜᵣₐₙₛ. The permanent shape is formed spontaneously and without further external influence. Cold-forming of a hairdo is a change of the hairdo at ambient temperature, without the use of additional heating by means of a hairdryer or similar devices. The deformation can, for example, be carried out mechanically, for example be means of subjecting the curls to gravity, by means of combing or brushing the hair, due to the influence of wind or humidity, due to mechanical influences during sleep or during lying.

The invention furthermore relates to a method for reprogramming a permanent hairdo, obtained with the above-mentioned method, into a different, new permanent shape. In order to achieve this goal, the initial hairdo is warmed to a temperature above T′ₜᵣₐₙₛ and the hair is brought into a new shape. Subsequently this new form is fixed by means of cooling to a temperature below T′ₜᵣₐₙₛ.

Shape memory polymers which may be crosslinked physically in accordance with the present invention are polymers, with which the fixation of the impressed permanent shape due to crosslinking occurs by physical interactions. A crosslinking by means of physical interactions can be achieved for example when specific segments of the polymer chains are assembled together in crystalline areas. The physical interactions may be charge transfer complexes, hydrogen bonds, dipolar or hydrophobic interactions, Van der Waals interactions or ionic interactions of polyelektrolyte segments. These interactions may occur between different segments within one polymer strand (intramolecular) and/or between different polymer strands (intermolecular). The formation of the interactions may be initiated by means of cooling (in particular in relation to crystallisation) and/or by means of drying, i.e. the removal of solvents.

Shape memory polymers which may be crosslinked physically and which are suitable in accordance with the present invention do show at least two transition temperatures Tₜᵣₐₙₛ and T′ₜᵣₐₙₛ. The two transition temperatures may for example be melting temperatures Tₘ or glass transition temperatures T_{g}. Above Tₜᵣₐₙₛ the polymer does show a lower modulus of elasticity compared with the modulus of elasticity below Tₜᵣₐₙₛ. The ratio of the moduli of elasticity below and above Tₜᵣₐₙₛ preferably is at least 10, more preferably at least 20. The lower transition temperature Tₜᵣₐₙₛ preferably is higher than room temperature (20° C.), in particular at least 30° C., more preferably at least 35° C. or at least 40° C. and this temperature is the temperature at which the spontaneous recovery of the permanent shape from the deformed or temporary shape occurs. Tₜᵣₐₙₛ is preferably above the usually to be expected ambient temperatures, so that at ambient temperatures no significant, undesired, thermally induced deformation of the temporary hairdo occurs. Suitable ranges for Tₜᵣₐₙₛ are for example from 25 to 100° C., from 30 to 75° C., from 35 to 70° C. or from 40 to 60° C.

The upper transition temperature T′ₜᵣₐₙₛ is higher than Tₜᵣₐₙₛ and this transition temperature is the temperature above which the impression of the permanent shape or the reprogramming of a permanent shape into a new permanent shape occurs and below which the permanent shape is fixed. T′ₜᵣₐₙₛ preferably is to the extend higher than Tₜᵣₐₙₛ, so that during the warming of the hairdo to a temperature above Tₜᵣₐₙₛ, for the recovery of the permanent shape or for the reforming of a temporary hairdo while maintaining the permanent shape, no significant, undesired, thermally induced deformation of the permanent shape occurs. Preferably, T′ₜᵣₐₙₛ is at least 10° C., more preferably at least 20° C. or at least 30° C. above Tₜᵣₐₙₛ. The difference between T′ₜᵣₐₙₛ and Tₜᵣₐₙₛ may be for example from 10 to 80° C., from 20 to 70° C. or from 30 to 60° C. Suitable ranges for T′ₜᵣₐₙₛ are for example from 40 to 150° C from 50 to 100° C. or from 70 to 95° C.

Suitable shape memory polymers which may be crosslinked physically are polymers which consist of at least one hard segment and at least one soft segment. The hard segment shows physical crosslinks and has a transition temperature T′ₜᵣₐₙₛ which is above room temperature, preferably more than 10° C. above 20° C. The soft segment has a transition temperature Tₜᵣₐₙₛ which is below T′ₜᵣₐₙₛ, for example at least 10° C. below T′ₜᵣₐₙₛ. The polymer segments are preferably oligomers, in particular linear chain molecules having a molecular weight of for example 400 to 30,000 preferably 1,000 to 20,000 or 1,500 to 15,000. These polymers may be linear di-block tri-block, tetra-block or multi-block copolymers, they may be branched, dendritic or graft copolymers. Preferably these polymers are not linear polyurethanes comprising bis(2-hydroxy-ethyl)-hydroquinone. The molecular weight of the polymers may for example be from 30,000 to 1,000,000, preferably from 50,000 to 700,000 or from 70,000 to 400,000. Suitable shape memory polymers which may be crosslinked physically are disclosed in WO 99/42147 and these polymers are disclosed there as thermoplastic polymers. The thermoplastic polymers disclosed in WO 99/42147 as well as the methods of preparation are suitable in accordance with the present invention and this disclosure is herewith incorporated by reference. These polymers do show a degree of crystallinity of preferably 3 to 80%, more preferably 3 to 60%. The ratio of the moduli of elasticity below and above Tₜᵣₐₙₛ preferably is at least 10, more preferably at least 20. The polymer segments may be segments derived from natural polymers, such as proteins or polysaccharides. The segments may also be synthetic polymer blocks. Suitable natural or synthetic polymer segments are identical with the crosslinkable macromers named above. Suitable shape memory polymers are in particular multiblock copolymers, comprising at least one first type of blocks and at least one different second type of blocks, wherein the blocks do enable that the multiblock copolymer possesses two different transition temperatures. Suitable multiblock copolymers are in particular copolymers prepared from at least two different macrodiols and at least one diisocyanate. Macrodiols are oligomers or polymers having at least two free hydroxyl groups. Oligomers do consist usually of at least two, preferably at least 3, more preferably 4 to 20, 5 to 15 or 6 to 10 monomers. The macrodiols may possess the general formula HO-A-OH, wherein A defines a divalent, oligomeric or polymeric group, preferably a polyester or an oligoester. The diisocyanate may have the general formula OCN—B—NCO, wherein B defines a divalent organic group, preferably an alkylene group or an arylene group, which may be further substituted. The alkylene group may be linear, branched or cyclic and this group preferably possesses 1 to 30 carbon atoms, more preferably 2 to 20 or 5 to 15 carbon atoms.

Particularly preferred shape memory polymers are the copolyester urethanes disclosed in WO 99/42147, in particular the reaction products of (a) two different macrodiols, selected among α, ω-dihydroxy polyesters, α, ω-dihydroxy oligoesters, α, ω-dihydroxy polylactones and α, ω-dihydroxy oligolactones, and (b) at least one diisocyanate, preferably trimethylhexane-1,6-diisocyanate. In particular preferred are macrodiols from poly(para-dioxanone) (PDX), poly(pentadecalactone) (PDL), poly(ε-caprolactone) (PCL), poly(L-lactide-co-glydolide) (PLGA). The molecular weights of the macrodiols are preferably within the range of from 400 to 30,000, preferable 1,000 to 20,000 or 1,500 to 15,000. The molecular weights of the resulting multiblock copolymers are preferably M_{w} from 30,000 to 1,000,000, more preferably from 50,000 to 70,000 or from 70,000 to 400,000 g/mol, as determined by GPC. The poly dispersity preferably is within the range of from 1.7 to 2.0.

Cationic Agents

Cationic agents are characterized in that they either carry at least one permanent cationic group within the molecule, for example an iminium group or an ammonium group, in particular a quaternary ammonium group, or that they carry at least one group which may be rendered cationic, for example a primary, secondary or tertiary amine group, which may be rendered cationic by the addition of a proton, wherein quaternary ammonium groups are preferred. The cationic agent is a compound which possesses an affinity to human hair due to the cationic group or the group which may be rendered cationic. Suitable cationic agents are for example surfactants having cationic groups or groups which can be rendered cationic, in particular cationic surfactants, betainic or amphoteric surfactants, polymers having cationic groups or groups which may be rendered cationic, in particular cationic, betainic or amphoteric polymers, silicone compounds having cationic groups or groups which may be rendered cationic, in particular diquaternary or polyquaternary siloxanes or Amodimethicone, cationic derivatives of proteins, cationic derivatives of protein hydrolysates or betaine.

Suitable cationic surfactants are surfactants which possess a quaternary ammonium group as well as a hydrophobic group. These surfactants may be cationic or amphoteric, betainic surfactants. Suitable cationic surfactants comprise amino groups or quaternary hydrophilic ammonium groups, which carry in solution a positive charge and these compounds can be described with the general formula (III).
N⁽⁺⁾R¹R²R³R⁴ X⁽⁻⁾ (III)
wherein R¹ to R⁴, independently, represent aliphatic groups, aromatic groups, alkoxy groups, polyoxyalkylene groups, alkyl amide groups, hydroxy alkyl groups, aryl groups or alkaryl groups having from 1 to 22 carbon atoms, wherein at least one residue possesses at least 8 carbon atoms, and wherein X⁻ represents an anion, for example a halogen, acetate, phosphate, nitrate or alkyl sulfate, preferably chloride. The aliphatic group may possess, in addition to the carbon atoms and the hydrogen atoms, also branches or other groups, such as for example amino groups.

Examples for suitable cationic surfactants are the chlorides or bromides of alkyl dimethyl benzyl ammonium salts, alkyl trimethyl ammonium salts, for example cetyltrimethyl ammonium chloride or bromide, tetradecyltrimethyl ammonium chloride or bromide, alkyl dimethyl hydroxyethyl ammonium chloride or bromide, the dialkyl dimethyl ammonium chlorides or bromides, alkyl pyridininium salts, for example lauryl or cetylpyridinium chloride, alkyl amido ethyl trimethyl ammonium ether sulfates, as well as compounds having a cationic character, such as amine oxides, for example alkyl methyl ammonium oxide or alkyl aminoethyl dimethyl amine oxide. In particular preferred is cetyltrimethyl ammonium chloride.

Suitable amphoteric surfactants are derivatives of aliphatic quaternary ammonium, phosphonium or sulfonium compounds of the formula (IV). 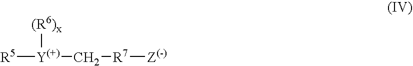
wherein R⁵ represents a linear or branched alkyl, alkenyl or hydroxy alkyl group having from 8 to 18 carbon atoms and 0 to about 10 ethylene oxide units and 0 to 1 glycerine units; Y represents a group containing N, B or S; R⁶ represents an alkyl or monohydroxyalkyl group with 1 to 3 carbon atoms; X=1 when Y represents a sulfur atom and X represents 2 when Y represents a nitrogen atom or a phosphorus atom; R⁷ represents an alkylene group or a hydroxy alkylene group having from 1 to 4 carbon atoms and Z⁽⁻⁾ represents a carboxylate, sulfate, phosphonate or phosphate group.

Other amphoteric surfactants, such as betaine, are as well suitable for the hair treatment composition in accordance with the present invention. Examples for betaine compounds comprise C8 to C18-alkylbetaine, such as Cocodimethylcarboxymethylbetain, Lauryldimethylcarboxymethylbetain, Lauryldimethylalphacarboxyethylbetain, Cetyldimethylcarboxy-methylbetain, Oleyldimethylgammacarboxypropylbetain und Lauryl-bis(2-hydroxypropyl)alphacarboxyethylbetain; C8- to C18-Sulfobetaine such as Cocodimethylsulfopropylbetain, Stearyl-dimethylsulfopropylbetain, Lauryldimethylsulfoethylbetain, Laurylbis-(2-hydroxyethyl)sulfopropylbetain; Carboxyl derivatives of Imidazol, C8- to C18-Alkyldimethyl-ammoniumacetate, C8- to C18-Alkyldimethylcarbonylmethylammonium salts as well as C8- to C18-Fatty acid alkylamidobetaines such as coco fatty acid amidopropylbetain und N-coco fatty acid amidoethyl-N-[2-(carboxymethoxy)ethyl]-glycerin (CTFA-name: Cocoamphocarboxyglycinate) and Cocamidopropyl Hydroxysultaine.

The suitable cationic polymers are preferably hair fixative or hair conditioning polymers. Suitable polymers comprise preferably quaternary amine groups. The cationic polymers may be homopolymers or copolymers, wherein the quaternary nitrogen groups are either comprised within the main chain or, preferably, as substituent attached to one or more of the monomers. The monomers comprising ammonium groups may be copolymerised with a non-cationic monomer. Suitable cationic monomers are unsaturated compounds which may be polymerised by means of a radical reaction, which comprise at least one cationic group, in particular ammonium substituted vinyl monomers, for example trialkyl methacryloxyalkyl ammonium, trialkyl acryloxyalkyl ammonium, dialkyl, diallyl ammonium and quaternary vinyl ammonium monomers having cyclic cationic groups comprising nitrogen, such as pyridinium, imidazolium or quaternary pyrrolidone, for example alkyl vinyl imidazolium, alkyl vinyl pyridinium or alkyl vinyl pyrrolidone salts. The alkyl groups of these monomers are preferably lower alkyl groups, such as C1 to C7 alkyl groups, more preferably C1 to C3 alkyl groups.

The monomers comprising ammonium groups may be copolymerised with non-cationic monomers. Suitable comonomers are for example acryl amide, methacryl amide, alkyl and dialkyl acryl amide, alkyl and dialkyl methacryl amide, alkyl acrylate, alkyl methacrylate, vinylcaprolacton, vinylcaprolactam, vinalpyrrolidone, vinylester, for example vinyl acetate, vinyl alcohol, propylene glycol or ethylene glycol, wherein the alkyl groups of these monomers comprise preferably C1 to C7 alkyl groups, more preferably C1 to C3 alkyl groups.

Suitable polymers having quaternary amine groups are for example the polymers disclosed in CTFA Cosmetic Ingredient Dictionary under the designation polyquaternium, for example Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, quaternized Vinylpyrrolidon/Dimethylaminoethylmethacrylate Copolymer (Polyquaternium-11), Polyquaternium-12, Poly-quaternium-13, Polyquaternium-14, Polyquaternium-15, Methyl-vinylimidazoliumchlorid/Vinylpyrrolidon Copolymer (Polyquaternium-16), Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, Polyquaternium-47, Polyquaternium-48, Polyquaternium-49, Polyquaternium-50, Polyquaternium-51, Polyquaternium-52, Polyquaternium-53, Polyquaternium-54, Polyquaternium-55, Polyquaternium-56.

Suitable are furthermore quaternary silicone polymers or oligomers, such as silicone polymers having quaternary terminal groups (quaternium-80). Of the cationic polymers, which may be contained in the composition in accordance with the present invention, the vinyl pyrrolidone/dimethyl amino ethyl methacrylate metho sulfate-copolymer, which is sold under the tradename Gafquat® 755 N and Gafquat® 734, of which Gafquat® 755 N is preferred, are suitable. Further cationic polymers are for example the copolymer of polyvinylpyrrolidone and imidazole imine methochloride, sold under the tradename LUVIQAUT® HM 550, the terpolymer of dimethyldiallyl ammonium chloride, sodium acrylate and acrylamide, sold under the tradename Merquat® Plus 3300, the terpolymer of vinylpyrrolidone, dimethyl amino ethyl methacrylate and vinylcaprolactam sold under the tradename Gaffix® VC 713, and the vinalpyrrolidone/methacrylamido propyl trimethyl ammonium chloride-copolymers sold under the tradename Gafquat® HS 100. Suitable are also cationic polyurethanes, for example formed from at least one organic diisocyanate and at least one organic dihydroxy compound substituted with at least one quaternary ammonium group; additionally non-ionic organic dihydroxy compounds may be copolymerised therewith.

Suitable cationic polymers, derived from natural polymers, are cationic derivatives of polysaccharides, for example cationic derivatives of cellulose, starch or guar. Suitable are furthermore chitosan and chitosan derivatives. Cationic polysaccharides have the general formula (V).
G-O—B—N⁺R^{a}R^{b}R^{c} X⁻ (V)
G represents an anhydroglucose residue, for example starch anhydroglucose or cellulose anhydroglucose; B represents a divalent linking group, for example alkylene, oxyalkylene, polyoxyalkylene or hydroxy alkylene; R^{a}, R^{b} and R^{c}, independently represent alkyl, aryl, alkylaryl, arylalkyl, alkoxyalkyl or alkoxyaryl having each up to 18 carbon atoms, wherein the total number of carbon atoms in R^{a}, R^{b} and R^{c}, preferably is at most 20; X represents a usual anion, which has the meaning as defined for formula (III) and X preferably represents chloride.

A cationic cellulose is sold under the tradename Polymer JR and has the INCI-designation polyquaternium-10. A further example of a cationic cellulose has the INCI designation polyquaternium-24 and is sold under the tradename Polymer LM-200. A suitable cationic guar derivative is sold under the tradename Jaguar R and has the INCI-designation guar hydroxypropyltrimonium chloride.

In particular preferred cationic agents are chitosan, chitosan salts and chitosan derivatives. The chitosans which may be employed in accordance with the present invention are completely or partially deacetylated chitins. For the preparation of chitosan the skilled person preferably starts with the chitin obtained from the shells of shell fish, which is available as inexpensive and natural raw material in large amounts. The molecular weight of the chitosan can cover a broad range, for example from 20,000 to 5,000,000 g/mol. Suitable is for example low molecular weight chitosan having a molecular weight of 30,000 to 70,000 g/mol. Preferably the molecular weight however exceeds 100,000 g/mol, in particular preferably the molecular weight ranges from 2000,000 to 700,000 g/mol. The degree of deacetylation preferably is 10 to 99%, more preferably 60 to 99%.

A suitable chitosan is for example sold under the tradename Flonac® and it has a molecular weight of from 300,000 to 700,000 g/mol and a degree of deacetylation of 70 to 80%. A preferred chitosan salt is chitosoniumpyrrolidone carboxylate, which is for example sold under the tradename Kytamer® PC. The chitosan contained therein has a molecular weight of about 200,00 to 300,000 g/mol and a degree of deacetylation of 70 to 85%. As chitosan derivatives quarternary, alkylated or hydroxy alkylated derivatives, for example hydroxyethyl or hydroxybutyl chitosan can be named. The chitosan or chitosan derivative preferably are present in neutralized or partially neutralized form. The degree of neutralization for the chitosan or the chitsan derivative preferably is at least 50%, more preferably between 70 and 100%, based on the number of free basic groups. As agent for the neutralization substantially all cosmetically acceptable inorganic or organic acids can be employed, such as formic acid, tartaric acid, malic acid, lactic acid, citric acid, pyrrolidone carboxylic acid, hydrochloric acid and the like, of which pyrrolidone carboxylic acid is in particular preferred.

Preferred are polymers which do possess a suitable water solubility or alcohol solubility, so that they may be present within the composition in accordance with the present invention in completely dissolved form. The cationic charge density preferably is from 1 to 7 meq/g.

Suitable cationic silicone compounds possess preferably either at least one amino group or at least one ammonium group. Suitable silicone polymers comprising amino groups are known under the INCI designation amodimethicone. These compounds are polydimethyl siloxanes having aminoalkyl groups. The aminoalkyl groups can be provided at the terminals or at the side chain positions. Suitable amino silicones do have the general formula (VI):
R⁸R⁹R¹⁰Si—(OSiR¹¹R¹²)x-(OSiR¹³Q)y-OSiR¹⁴R¹⁵R¹⁶ (VI)
R⁸, R⁹, R¹⁴ and R¹⁵, independently, are identical or different and each represent C1 to C10 alkyl, phenyl, hydroxy, hydrogen, C1 to C10 alkoxy or acetoxy, preferably C1 to C4 alkyl, in particular methyl; R¹⁰ and R¹⁶, independently, are identical or different and represent —(CH₂)ₐ—NH₂, wherein a represents an integer from 1 to 6, C1 to C10 alkyl, phenyl, hydroxy, hydrogen, C1 to C10 alkoxy or acetoxy, preferably C1 to C4 alkyl, in particular methyl; R¹¹, R¹² and R¹³, independently, are identical or different and represent hydrogen, C1 to C20 hydrocarbon, which may comprise oxygen and nitrogen, preferably C1 to C10 alkyl or phenyl, in particular C1 to C4 alkyl, in particular methyl; Q represents -A-NR¹⁷R¹⁸ or -A-N⁺R¹⁷R¹⁸R¹⁹, wherein A represents a divalent C1 to C20 alkylene linking group, which may comprise oxygen and nitrogen as well as hydroxyl groups, and wherein R¹⁷ R¹⁸ R¹⁹, independently, are identical or different and represent hydrogen, C1 to C22 hydrocarbon, preferably C1 to C4 alkyl or phenyl. Preferred residues for Q are
(CH₂)₃—NH₂, —(CH₂)₃NHCH₂CH₂NH₂, —(CH₂)₃OCH₂CHOHCH₂NH₂ and —(CH₂)₃N(CH₂CH₂OH)₂, —(CH₂)₃—NH₃⁺ and —(CH₂)₃OCH₂CHOHCH₂N⁺(CH₃)₃R²⁰, wherein R²⁰ represents a C1 to C22 alkyl group, which may comprise hydroxyl groups. X represents a number between 1 and 10,000, preferably between 1 and 1000; and Y represents a number between 1 and 500, preferably between 1 and 50.

The molecular weight of the amino silicone compounds preferably lies between 500 and 100,000. The amine content (meq/g) preferably lies within the range of from 0.05 to 2.3, preferably 0.1 to 0.5.

Suitable silicone polymers having two terminal quaternized ammonium groups are known under the INCI-designation Quaternium-80. These polymers are dimethoxysiloxanes having two terminal aminoalkyl groups. Suitable quarternary aminosilicones have the following general formula (VII)
R²¹R²²R²³N⁺-A-SiR⁸R⁹—(OsiR¹¹R¹²)ₙ—OsiR⁸R⁹-A-N³⁰R²¹R²²R²³ 2X⁻ (VII)

A has the meaning as defined above for formula (VI) and preferably represents —(CH₂)₃OCH₂CHOHCH₂N⁺(CH₃)₂R²⁰, wherein R²⁰ represents a C1 to C22 alkyl group, which may also comprise —OH groups; R⁸, R⁹, R¹¹ and R¹² have the meaning as defined above for formula (VI), preferably methyl; R²¹, R²², R²³, independently, represent C1 to C22 alkyl groups, which may comprise hydroxyl groups, and wherein preferably at least one of the residues comprises at least 10 carbon atoms and the others comprise 1 to 4 carbon atoms; n represents a number from 0 to 200, preferably from 10 to 100. Quarternary polydimethyl siloxanes of this type are sold under the designation Abil© Quat 3270, 3272 and 3274.

Further suitable cationic hair conditioning compounds are cationically modified protein derivatives or cationically modified protein hydrolysates and these are for example known under the INCI-designation Lauryldimonium Hydroxypropyl Hydrolyzed Wheat Protein, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein oder Hydroxypropyltrimonium Hydrolyzed Wheat, Hydroxypropyltrimonium Hydrolyzed Casein, Hydroxypropyltrimonium Hydrolyzed Collagen, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyltrimonium Hydrolyzed Vegetable Protein.

Suitable cationically modified protein hydrolysates are mixtures which may for example be obtained after reacting proteins, hydrolysed by a basic, acidic or enzyme reaction, with glycidyl trialkyl ammonium salts or 3-halo-2-hydroxy propyl trialkyl ammonium salts. Proteins which may serve as starting materials for the protein hydrolysates are of vegetal or animal origin. Suitable starting materials are keratin, collagen, elastin, soy proteins, rice proteins, milk proteins, wheat proteins, silk proteins or almond proteins. Due to the hydrolysis mixtures are obtained comprising compounds having molecular weights in the range of about 100 to about 50,000. Typical average molecular weights are in the range of about 500 to about 1,000. Preferably the cationically modified protein hydrolysates contain one or two long C8 to C22 alkyl chains and, corresponding thereto two or one short C1 to C4 alkyl chains. Compounds, which comprise one long alkyl chain are prefered.

Compositions according to the present invention for the treatment of hair comprise the first active principle, which alone or in combination with other compounds is responsible for the shape memory effect, in an amount of preferably from 0.01 to 25 wt.-%, more preferably 0.1 to 15 wt.-%, in a suitable medium. Preferred amounts of the cationic agent are 0.01 to 10 wt.-%, more preferably 0.05 to 5 wt.-%. The Composition may be present as solution, dispersion, emulsion, suspension or latex. The liquid, gel-type, half-solid or solid medium is substantially cosmetically and physiolically acceptable.

The composition in accordance with present invention usually is present in the form of a solution or dispersion comprising a suitable solvent. Preferred are in particular aqueous, alcoholic or aqueous-alcoholic solvents. Suitable solvents are, e.g., aliphatic alcohols having from 1 to 4 carbon atoms or a mixture of water with at least one of these alcohols. Other organic solvents can, however, also be employed, in particular linear or branched hydrocarbons, such as pentane, hexane, isopentan, cyclic hydrocarbons such as cyclopentane and cyclohexan, organic linear or cyclic ethers, such as tetrahydrofurane (THF) or liquid organic esters, such as ethylacetate. Furthermore, solvents on the basis of silicone materials are suitable, in particular silicone oils on the basis of linear or cyclic polydimethylsiloxanes (dimethicone or Cyclomethicone). Preferred are volatile silicones having a boiling point of below 200° C. Additional solvents are acetone, THF, trichloromethane etc. The solvents are preferably present in an amount from 0,5 to 99 weight %, more preferably in an amount from 10 to 97wt.-%, more preferably 20 to 95 wt.-% or 40 to 90 weight %.

The compositions in accordance with present invention may comprise in addition from 0.01 to 25 weight % of at least one hair fixative, hair coloring agent and/or hair cosmetic. Hair fixatives are in particular the known conventional film-forming hair fixative polymers. The film forming and hair fixative polymer may be of synthetic origin or of natural origin and may be of nonionic, cationic, anionic or amphoteric character. Such a polymer additive, which can be present in an amount of from 0.01 to 25 weight %, preferably 0.1 to 20 weight %, more preferably 0.5 to 15 weight %, may also comprise a mixture of more than one polymers, and this additive can be modified further with respect to the hair fixative properties by means of the addition of further polymers having a thickening effect. Film-forming, hair fixative polymers in accordance with present invention are polymers which are able, when employed in aqueous, alcoholic or aqueous-alcoholic solutions at a concentration of from 0.01 to 5%, to provide a polymer film on the hair in order to provide fixative properties. As suitable, synthetic, nonionic, film-forming, hair fixative polymers, the hair treatment composition in accordance with present invention may comprise homo polymers of vinyl pyrrolidone, homopolymers of N-vinylformamide, copolymers of vinylpyrrolidone and vinylacetate, terpolymers of vinylpyrrolidone, vinylacetate and vinylpropionate, polyacrylamide, polyvinylalcohols, or polyethyleneglycols having a molecular weight of from 800 to 20.000 g/mol. Suitable, synthetic, anionic, film-forming polymers are crotonic acid/vinylacetate copolymers and terpolymers of acrylic acid, ethylacrylate and N-t-butylacrylamide. Natural film-forming polymers or polymers obtained therefrom by means of chemical derivatization may also be employed in the hair treatment composition in accordance with present invention, for example China balsam resin, cellulose derivatives such as hydroxypropylcellulose having a molecular weight of from 30,000 to 50,000 g/mol, or shellac in its neutralized or unneutralized form. Amphoteric polymers can also be employed in the hair treatment composition in accordance with present invention. Suitable are, e.g., copolymers of octylacrylamide, t-butylaminoethylmethacrylate and two or more monomers selected from the group consisting of acrylic acid, methacrylic acid and simple esters thereof.

The consistency of hair treatment compositions in accordance with present invention can be improved by means of the addition of thickeners. In this respect, homopolymers of acrylic acid having a molecular weight of 2,000,000 to 6,000,000 g/mol are suitable. Further copolymers of acrylic acid and acrylamide (sodium salt) having a molecular weight of from 2,000,000 to 6,000,000 g/mol, sclerotium gum and copolymers of acrylic acid and methacrylic acid are suitable.

A cosmetic composition in accordance with present invention can be employed in the form of different formulations for different types of applications, e.g., as lotion, as spray lotion, as cream, as gel, as foam-gel, as aerosol spray, as non-aerosol spray, as aerosol foam, as non-aerosol foam, as o/w- or w/o-emulsion, as micro emulsion or as hair wax.

When the hair treatment composition in accordance with present invention is provided in the form of an aerosol spray, the composition comprises additionally from 15 to 85 weight %, preferably from 25 to 75 weight % of a propellant and the composition is filled into a pressurized canister having a spray button As propellants, lower alkanes, such as n-butane, isobutene and propane as well as mixtures thereof as well as dimethylether or fluorohydrocarbons, such as F 152a (1,1-difluorethane) or F 134 (tetrafluorethane) are suitable, as well as propellants which are present at the pressures in question in gaseous forms such as N₂, N₂O and CO₂ as well as mixtures of the above-mentioned propellants.

When the hair treatment composition of the present invention is provided in the form of a sprayable non-aerosol hair spray, the composition is sprayed by means of a suitable, mechanical spraying device. Spray devices of the mechanical type are devices which allow the spraying of a composition without using a propellant. Suitable mechanical spray devices are, e.g., spray pumps or elastic containers provided with a spray valve, into which the cosmetic composition in accordance with present invention is filled under pressure, whereby the elastic container is expanded and wherein the cosmetic composition is continuously discharged if the valve is open, due to the contraction of the elastic container.

When the hair treatment composition in accordance with present invention is provided in the form of a hair foam (mousse), the composition comprises at least one conventional foaming agent known in the art for this purpose. The composition is foamed with or without the aid of propellants or chemical propellants and the foam may be applied to the hair and may remain in the hair without the need of rinsing the hair. A product in accordance with present invention comprises as additional component a device for the provision of a foam of the composition. Devices for providing a foam are devices which allow the provision of a foam starting from a liquid, with or without the use of a propellant. Suitable mechanical foaming devices are, e.g., usual foam pumps, or usual aerosol foaming heads.

When the hair treatment composition in accordance with present invention is provided in the form of a hair gel, it comprises at least one gel forming substance in an amount of preferably from 0.05 to 10, more preferably from 0.1 to 2 weight %. The viscosity of the gel preferably amounts to from 100 bis 50,000 mm²/s, more preferably 1,000 bis 15,000 mm²/s at 25° C., measured as dynamic viscosity using a Bohlin Rheometer CS, measurement body C25 using a shear velocity of 50 s⁻¹.

When the hair treatment composition in accordance with present invention is provided in the form of a hair wax, it comprises additionally water-soluble fatty substances or waxy substances, or substances which provide the composition with a waxy consistence, in an amount of preferably 0.5 bis 30 weight %. Suitable water unsoluble substances are, for example, emulgators having a HLB-value of below 7, silicone oils, silicone waxes, wax materials (e.g., waxy alcohols, waxy acids, waxy esters, as well as natural waxes such as beeswax, Carnauba wax, etc.), fatty alcohols, fatty acids, fatty acid esters or high molecular polyethyleneglycols having a molecular weight of from 800 bis 20,000, preferably from 2,000 to 10,000 g/mol.

When the hair treatment composition in accordance with present invention is provided in the form of a hair lotion, it is present as a substantially non-viscous or low viscosity, flowable solution, dispersion or emulsion comprising a content of at least 10 weight %, preferably 20 to 95 weight % of a cosmetically acceptable alcohol. As alcohols, the usual alcohols used for cosmetic purposes can be named, in particular the lower C1 to C4 alcohols such as ethanol and isopropanol.

When the hair treatment composition of the present invention is present in the form of a hair cream, it is preferably provided in the form of an emulsion and it either comprises additional viscosity providing components in an amount of from 0.1 to 10 weight % or the required viscosity and creamy consistency is obtained by means of micelle formation using suitable emulsifiers, fatty acids, fatty alcohols, waxes, etc.

In a preferred embodiment the composition in accordance with present invention is provided in a form enabling, at the same time, the impression of a recallable hairdo as well as providing hair coloration. The composition is then formulated as coloring hair treatment composition, e.g., as coloring fixative, coloring cream, coloring foam, etc. It comprises at least one coloring component. The coloring component may be an organic dye, in particular direct dyes or it may be an inorganic pigment.

The total amount of coloring agent amounts in the composition in accordance with present invention to about 0.01 to 7 weight %, preferably about 0.2 to 4 weight %. Suitable direct colorants to be used in the composition in accordance with present invention are, e.g., triphenylmethane colorants, aromatic nitro colorants, azo colorants, quinone colorants, cationic or anionic colorants.

Suitable hair colouring pigments are substantially insoluble in the medium of the composition and these pigments may be of organic or inorganic nature. Organic-inorganic mixed phase pigments are also suitable. The pigments preferably are not nanopigments. The preferred particle size is 1 to 200 μm, preferably 3 to 150 μm, more preferably 10 to 100 μm. Preferred are inorganic pigments.

The hair treatment composition in accordance with the present invention preferably comprises additionally at least one hair cosmetic in an amount of from 0.01 to 10, more preferably of from 0.05 to 5 weight %. Preferred hair cosmetics are oily compounds, such as mineral oils, silicone oils, oily fatty acid esters, oils of vegetable origin etc.

Suitable silicone compounds are for example polydimethylsiloxane (INCI: Dimethicone), α-hydro-ω-hydroxypolyoxydimethylsilylene (INCI: Dimethiconol), cyclic dimethylpolysiloxane (INCI: cyclomethicone), trimethyl(octadecyloxy)silane (INCI: stearoxytrimethylsilane), dimethylsiloxane/glycol copolymer (INCI: dimethicone copolyol), dimethylsiloxane/aminoalkylsiloxane copolymer having terminal hydroxy groups (INCI: amodimethicone), monomethylpolysiloxane having lauryl side chains and terminal polyoxyethylene chains and/or polyoxypropylene chains (INCI: lauryl methicone copolyol), dimethylsiloxane/glycol copolymer acetate (INCI: dimethiconcopolyol acetate), dimethylsiloxane/aminoalkylsiloxane copolymer having terminal trimethylsilyl groups (INCI: trimethylsilylamodimethicone). Preferred silicone polymers are dimethicone, cyclomethicone and dimethiconole. Mixtures of silicone polymers are also suitable, for example a mixture of dimethicone and dimethiconol. The designations provided in brackets correspond to the nomenclature in accordance with INCI (International Cosmetic Ingredients), which is employed for the designation of cosmetically relative active ingredients and additives.

Usually further known cosmetic additives may be added to the hair treatment composition in accordance with the present invention, for example non-fixative non-ionic polymers, such as polyethylene glycols, non-fixative, anionic and natural polymers as well as mixtures thereof, in amounts of preferably from 0.01 to 50 weight %. Perfume oils in amount of from 0.01 to 5 weight %, opacifier such as ethyleneglycoldistearate in an amount of from 0.01 to 5 weight %, surfactants or emulsifiers selected from the classes of anionic, cationic, amphoteric or non-ionic surfactants, such as fatty alcohol sulfates, ethoxylated fatty alcohols, fatty acid alcanolamides, such as esters of hydrated fatty acids from rizinus oil, in an amount of from 0.1 to 30 weight %, as well as moisturizers, dye receptivity improving agents, light protective agents, anti-oxidative agents as well as preservatives in amounts of from 0.01 to 10 weight % may be added.

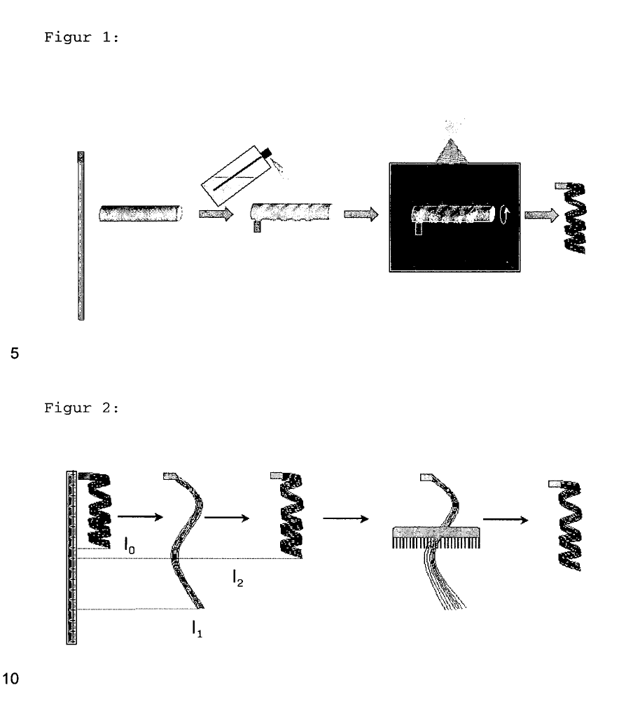

FIG. 1 shows schematically the method for the provision of a recallable, permanent hairdo. A strand of hair is wound onto a curler (bobbin) and a solution in accordance with the present invention comprising a cross-linkable macromer is sprayed thereon. By irradiation with a suitable source of energy, for example a UV lamp, the desired permanent shade is fixed. Subsequently the curler is removed.

FIG. 2 shows the deformation of a permanent hairdo and the recovery of the permanent shape starting from the temporary shape. The hair curl in the permanent shape possesses a length l₀. The curl in the deformed shape has a length l₁. The curl in the recovered shape has a length l₂. The degree of re-attaining the permanent shape (recovery) can be calculated as follows: Recovery=(l₁−l₂)/(l₁−l₀).

As measure for the evaluation of the shape memory property of a composition the memory factor may be employed, which considers the transformation ability of a permanent hairdo into a temporary shape (shaping factor) as well as the recovery attainability of the permanent form starting from the temporary form (recovery factor, degree of recovery). If one starts with the consideration from an even strand of hair onto which a curly shape has been impressed as permanent shape and onto which, subsequently, a second, even shape has been impressed as temporary shape, the shaping factor can be determined in accordance with the following criterion:

| | Shaping |
|---|---|
| Degree of evenness | factor |
| Strong curl from the hairline to the end of hair | 0 |
| Lesser degree of curliness from the hairline to | 1 |
| the end of hair | |
| Hairline even, end of hair in the form of a curl | 2 |
| Hairline even, slight curvature at the end of | 3 |
| hair | |
| Even from the end of hairline to the end of hair | 4 |

The recovery factor can be determined in accordance with the following criterion:

| | Recovery |
|---|---|
| | (memory) |
| Degree of recovery of the permanent shape | factor |
| 0% | 0 |
| 30% | 1 |
| 40% | 2 |
| 50% | 3 |
| 60% | 4 |
| 75% | 5 |
| 100% | 6 |

The memory factor M can be calculated using the shaping factor F, the maximum shaping factor F=4, the recovery factor r and the maximum recovery factor R=6 in accordance with the following equation
M=(f/F)*(r/R)*100

The memory factor should ideally be not below 25, preferably the factor should be between 25 and 33.3, more preferred between 37 and 100.

The following examples are intended to illustrate the subject matter of the present invention further.

### EXAMPLES

### Examples 1-6

Compositions according to table 1 were prepared (amounts are given in gramm)

TABLE 1

| | 1 | 2 | 3 | 4 | 5 | 6 |
|---|---|---|---|---|---|---|
| PEG-8k- | 1.5 | 2.7 | 2.7 | 2.7 | 2.7 | — |
| DMA¹⁾ | | | | | | |
| PEG-8k^{1a)} | — | — | — | — | — | 1.5 |
| Poly- | 0.1 | — | — | — | — | 0.1 |
| quaternium-56 | | | | | | |
| Celquat ® | — | 0.3 | — | — | — | — |
| L200²⁾ | | | | | | |
| Gafquat ® | — | — | 0.3 | — | — | — |
| 755N³⁾ | | | | | | |
| Cationic | — | — | — | 0.3 | — | — |
| terpolymer⁴⁾ | | | | | | |
| Aquaflex ® | | | | | 0.3 | |
| SF 40⁵⁾ | | | | | | |
| Ethanol/ | Ad 100 | Ad 100 | Ad 100 | Ad 100 | Ad 100 | Ad 100 |
| Water 1:1 | | | | | | |
| Recovery | 50 | 38 | 30 | 40 | 33 | 33 |
| (Memory) | | | | | | |
| Factor | | | | | | |

¹⁾Polyethylenglycol having a molecular weight of 8000, terminal methacrylic acid groups
^{1a)}Polyethylenglycol having a molecular weight of 8000
²⁾cationic cellulose, Polyquaternium-4
³⁾Polyquaternium-11
⁴⁾cationic copolymer of hydroxy ethyl methacrylat/quaternized dimethyl amino ethyl methacrylat/dodecyl methacrylat, obtainable by radical copolymerization of the comonomers with AIBN in a ratio of 6/77/17.
⁵⁾Vinylcaprolactam/Vinylpyrrolidon/Dimethylaminopropylmethacrylate Copolymer

The transition temperatures Tₜᵣₐₙₛ of the shape memory sytems were in the range of 55-57° C. The memory factor M was determined in accordance with the method disclosed above.

For the preparation of a permanent shape a strand of hair havin a length of 20 cm, moistened with water, was wound onto a curler and the polymer solution was applied thereon (20 to 30 mg polymer/g hair). The treated strand of hair is then fixed at 70° C. for 30 minutes with application of UV light (examples 1 to 5) or without application of UV light (example 6). After cooling to room temperature (about 25° C.) the curler is removed. The curled strand (impressed permanent shape) had a length of about 4.5 cm. In order to prepare a temporary shape (e.g. even strand) the curled strand is warmed to about 55° C., extended to the original length (20 cm) and cooled again to room temperature. The permanent shape can be recovered by warming the even strand to about 55° C. The strand retracted at this temperature sponaneously to the permanent (curly) shape. In order to obtain again the temporary (e.g. even) shape the curled strand again is warmed to about 55° C., extended to the full length (20 cm) and cooled to room temperature.

### Examples 7 to 16 Using Cross-Linkable Macromers

The following hair treatment compostions were prepared using the macromers listed below which can be cross-linked in order to provide shape memory polymers. The preparation of the macromers was carried out as described in WO 99/42147.
M1 PEG(4 k)-DMA, Polyethylenglycol having a molecular weight of about 4000, twice esterified with methacrylic acid
M2 PEG(8 k)-DMA, Polyethylenglycol having a molecular weight of about 8000, twice esterified with methacrylic acid
M3 PEG(10 k)-DMA, Polyethylenglycol having a molecular weight of about 10000, twice esterified with methacrylic acid
M4 PLGA(7 k) -DMA, Poly(L-lactid-co-glycolid)-dimethacrylate having a molecular weight of about 7000
M5 PCl(10 k)-DMA, Poly(ε-caprolactone)-dimethacrylate having a molecular weight of about 10000

The application of the following hair treatment compositions was carried out as disclosed in example 1 with similar results.

### Example 7

### Hair Fixative

| | | |
|---|---|---|
| 2 | g | Macromer M1 |
| 1.50 | g | Vinylpyrrolidone/Vinylacetate Copolymer |
| 0.20 | g | 1,2-Propyleneglycol |
| 0.15 | g | Perfume |
| 0.5 | g | Cetyltrimethylammoniumchlorid |
| 45 | g | Water |
| Ad 100 | g | Ethanol |

### Example 8

### Pump-Spray Lotion

| | | |
|---|---|---|
| 2 | g | Macromer M2 |
| 0.2 | g | Polyquaternium-16 |
| 0.4 | g | Chitosane |
| 2.5 | g | PVP/VA Copolymer |
| 0.12 | g | Formic aid |
| 0.15 | g | Cetrimoniumphosphat |
| 0.1 | g | Laureth-4 |
| 0.1 | g | 2-Hydroxy-4-methoxybenzophenone |
| 0.1 | g | Perfume |
| 50 | g | Ethanol |
| Ad 100 | g | Water |

### Example 9

### Aerosol-Foam

| | | |
|---|---|---|
| 2 | g | Macromer M3 |
| 2.00 | g | Polyquaternium-11 (Gafquat ® 755) |
| 0.45 | g | Glyceryllaurat |
| 0.15 | g | Perfume |
| 0.16 | g | Cetyltrimethylammoniumchloride |
| 5.00 | g | Propane/Butane (5.0 bar) |
| 15 | g | Ethanol |
| Ad 100 | g | Water |

### Example 10

### Aerosol-Foam

| | | |
|---|---|---|
| 2 | g | Macromer M4 |
| 3.40 | g | Vinylcaprolactame/Vinylpyrrolidone/Dimethyl- |
| | | aminoethylmethacrylate Terpolymer |
| 0.60 | g | Formic acid |
| 0.60 | g | Hydrogenated castor oil, ethoxylated with 40 |
| | | Mol Ethyleneoxide |
| 0.22 | g | Decylpolyglucoside |
| 0.09 | g | Cetyltrimethylammoniumchloride |
| 0.20 | g | Perfume |
| 6.00 | g | Propane/Butane (5.0 bar) |
| Ad 100 | g | Water |

### Example 11

### Aerosol-Spray

| A | | B | | C | | |
|---|---|---|---|---|---|---|
| 2 | g | | | | | Macromer M1 |
| | | 2 | g | | | Macromer M2 |
| | | | | 2 | g | Macromer M5 |
| 1.50 | g | 1.50 | g | 1.50 | g | Octylacrylamide/Acrylic |
| | | | | | | acid/Butyl- |
| | | | | | | aminoethylmethacrylate/ |
| | | | | | | Methacrylate/ |
| | | | | | | Hydroxypropylmethacrylate |
| | | | | | | Copolymer (Amphomer ®) |
| 0.15 | g | 0.15 | g | 0.15 | g | Perfume |
| 10.67 | g | 10.67 | g | 10.67 | g | Butane (1.5 bar) |
| 33.33 | g | 33.33 | g | 33.33 | g | Propane/Butane |
| Ad 100 | g | Ad 100 | g | Ad 100 | g | Ethanol |

### Example 12

### Pumpspray

| A | | B | | |
|---|---|---|---|---|
| 2 | g | | | Macromer M3 |
| | | 2 | g | Macromer M4 |
| 1 | g | 1 | g | Octylacrylamide/Acrylic |
| | | | | acid/Butyl- |
| | | | | aminoethylmethacrylate/Methacrylate/ |
| | | | | Hydroxypropylmethacrylate |
| | | | | Copolymer (Amphomer ®) |
| 0.3 | g | 0.3 | g | Perfume |
| 0.1 | g | 0.1 | g | PEG-12 Dimethicone |
| 10 | g | 10 | g | Water |
| Ad 100 | g | Ad 100 | g | Ethanol |

### Example 13

### Aerosol Coloring Foam

| | | |
|---|---|---|
| 2 | g | Macromer M5 |
| 1.00 | g | Polyquaternium-11 (Luviquat ® PQ 11) |
| 0.11 | g | 3-(((2-Nitro-4-(trifluormethyl)phenyl)amino)- |
| | | 1,2-propandiol |
| 0.20 | g | 1,2-Propyleneglycol |
| 0.17 | g | Perfume |
| 0.10 | g | Cetyltrimethylammoniumchloride |
| 6.00 | g | Propane/Butane (5.0 bar) |
| 18.66 | g | Ethanol |
| Ad 100 | g | Water |

### Example 14

### Coloring Foam Fixative

| | | |
|---|---|---|
| 2 | g | Macromer M1 |
| 1.00 | g | Polyquaternium-11 (Gafquat ® 755) |
| 20.10 | g | Colorona Carmine Red |
| 0.20 | g | 1,2-Propyleneglycol |
| 0.17 | g | Perfume |
| 0.10 | g | Cetyltrimethylammoniumchloride |
| 6.00 | g | Propane/Butane (5.0 bar) |
| 18.66 | g | Ethanol |
| Ad 100 | g | Water |

### Example 15

### Pump-Foam Fixative

| | 1. A | | 2. B | | C | | D | |
|---|---|---|---|---|---|---|---|---|
| Macromer M2 | 2 | g | | | | | | |
| Macromer M3 | | | 2 | g | | | | |
| Macromer M4 | | | | | 2 | g | | |
| Macromer M5 | | | | | | | 2 | g |
| PVP (K80) | — | | — | | 1.4 | g | 1.4 | g |
| Vinyl- | 1.4 | g | 0.9 | g | — | | — | |
| caprolactame/ | | | | | | | | |
| PVP/DMAPA | | | | | | | | |
| Copolymer | | | | | | | | |
| Cocamido- | 0.6 | g | 0.6 | g | 0.6 | g | 0.6 | g |
| propyl | | | | | | | | |
| Hydroxy- | | | | | | | | |
| sultaine | | | | | | | | |
| Cetrimonium- | 0.25 | g | 0.25 | g | 0.25 | g | 0.25 | g |
| chloride | | | | | | | | |
| Betaine | 0.1 | g | 0.1 | g | 0.1 | g | 0.1 | g |
| Citric acid | 0.1 | g | 0.1 | g | 0.1 | g | 0.1 | g |
| Perfume | 0.15 | g | 0.15 | g | 0.15 | g | 0.15 | g |
| Ethanol | 2.5 | g | 2.5 | g | 2.5 | g | 2.5 | g |
| Water | Ad 100 | g | Ad 100 | g | Ad 100 | g | Ad 100 | g |

### Example 16

### Aerosol-Foam

| A | | B | | C | | |
|---|---|---|---|---|---|---|
| 2 | g | | | | | Macromer M1 |
| | | 2 | g | | | Macromer M2 |
| | | | | 2 | g | Macromer M3 |
| 0.6 | g | 0.6 | g | 0.6 | g | Cyclomethicone |
| 0.5 | g | 0.5 | g | 0.5 | g | Propyleneglycol |
| 0.5 | g | 0.5 | g | 0.5 | g | Quaternium-80; |
| | | | | | | diquaternary |
| | | | | | | Dimethylsiloxane |
| 0.2 | g | 0.2 | g | 0.2 | g | Cetyltrimethyl- |
| | | | | | | ammoniumchloride |
| 0.2 | g | 0.2 | g | 0.2 | g | Polyquaternium-11 |
| 0.1 | g | 0.1 | g | 0.1 | g | PEG-12 Dimethicone |
| 6 | g | 6 | g | 6 | g | Propane/Butane |
| Ad 100 | g | Ad 100 | g | Ad 100 | g | Water |

The macromers used in examples 7-16 may be replaced, partially or in total, with any of the macromers M1 to M17, as alternative it is also possible to add one or more of the shape memory polymers defined below as P1 to P8, with similar results.

### Examples 17-26

### Hair Treatment Using Thermoplastic Shape Memory Polymers

The shape memory polymers used in the following examplary cosmetic compositions can be prepared from two different macrodiols and trimethylhexan-1,6-diisocyanate in accordance with the procedure disclosed in example 1 of WO 99/42147.

| Macrodiol | P1 | P2 | P3 | P4 |
|---|---|---|---|---|
| PDX 1500 [wt. %] | 48 | | | |
| PLGA 2000 [wt. %] | 52 | | | |
| PCL 4000 [wt. %] | | 60 | | |
| PCL 10000 [wt. %] | | | 50 | 60 |
| PDL 2000 [wt. %] | | 40 | | |
| PDL 3000 [wt. %] | | | 50 | 40 |
| M_{w} Copolymer | 79100 | 150000 | 130000 | 115000 |
| [g/mol] | | | | |
| Tₜᵣₐₙₛ [° C.] | 34 | 48 | 55 | 55 |
| T′ₜᵣₐₙₛ [° C.] | 85 | 89 | 87 | 87 |

| Macrodiol | P5 | P6 | P7 | P8 |
|---|---|---|---|---|
| PCL 10000 [wt. %] | 78 | 59 | 40 | 20 |
| PDL 10000 [wt. %] | 22 | 41 | 60 | 80 |
| M_{w} Copolymer | 208000 | 357000 | 282000 | 300100 |
| [g/mol] | | | | |
| Tₜᵣₐₙₛ [° C.] | 55 | 54 | 55 | 54 |
| T′ₜᵣₐₙₛ [° C.] | 91 | 91 | 93 | 94 |

The abbreviations of the macrodiols have the following meaning:
PDX: Poly(para-dioxanone)
PLGA: Poly(L-lactide-co-glycolide)
PCL: Poly(ε-caprolactone)
PDL: Poly(pentadecalactone)

The numbers presented in connection with the macrodiols represent the approx. molecular weight of the macrodiols (±100).

The following hair treatment compositions are used as follows:

A sufficient amount, depending from the length of the hair, of the composition is applied onto the hair. The hair is brought into the desired shape, for example using curlers onto which the hair is wound, or by stretching the hair, followed by drying. Subsequently the hair is warmed to about 95° C. After cooling to room temperature (about 25° C.) the curlers are removed. Impressing a second shape of a hairdo (temporary shape) is achieved by warming the first hairdo to about 55° C., bringing the hair into a desired second form of hairdo and cooling to room temperature. Rewarming to about 55° C. enables the spontaneous recovery of the first hairdo.

### Example 17

### Hair Fixative

| | | |
|---|---|---|
| 0.5 | g | Polymer P1 |
| 1.50 | g | Vinylpyrrolidone/Vinylacetatee Copolymer |
| 0.20 | g | 1,2-Propyleneglycol |
| 0.15 | g | Perfume |
| 0.5 | g | Cetyltrimethylammoniumchloride |
| 45 | g | Water |
| Ad 100 | g | Ethanol |

### Example 18

### Pump-Spray Lotion

| | | |
|---|---|---|
| 1 | g | Polymer P2 |
| 0.2 | g | Polyquaternium-16 |
| 0.4 | g | Chiosane |
| 2.5 | g | PVP/VA Copolymer |
| 0.12 | g | Formic acid |
| 0.15 | g | Cetrimoniumphosphate |
| 0.1 | g | Laureth-4 |
| 0.1 | g | 2-Hydroxy-4-methoxybenzophenone |
| 0.1 | g | Perfume |
| 50 | g | Ethanol |
| Ad 100 | g | Water |

### Example 19

### Aerosol-Foam

| | | |
|---|---|---|
| 1.5 | g | Polymer P3 |
| 2.00 | g | Polyquaternium-11 (Gafquat ® 755) |
| 0.45 | g | Glyceryllaurat |
| 0.15 | g | Perfume |
| 0.16 | g | Cetyltrimethylammoniumchloride |
| 5.00 | g | Propane/Butane (5.0 bar) |
| 15 | g | Ethanol |
| Ad 100 | g | Water |

### Example 20

### Aerosol-Foam

| | | |
|---|---|---|
| 2 | g | Polymer P4 |
| 3.40 | g | Vinylcaprolactame/Vinylpyrrolidone/Dimethyl- |
| | | aminoethylmethacrylate Terpolymer |
| 0.60 | g | Formic acid |
| 0.60 | g | Hydrogenated castor oil, ethoxylated with 40 Mol |
| | | Ethyleneoxide |
| 0.22 | g | Decylpolyglucoside |
| 0.09 | g | Cetyltrimethylammoniumchloride |
| 0.20 | g | Perfume |
| 6.00 | g | Propane/Butane (5.0 bar) |
| Ad 100 | g | Water |

### Example 21

### Aerosol-Spray

| | | |
|---|---|---|
| 0.5 | g | Polymer P5 |
| 1.50 | g | Octylacrylamide/Acrylic |
| | | acid/Butylaminoethylmethacrylate/Methacrylate/ |
| | | Hydroxypropylmethacrylate Copolymer |
| 0.15 | g | Perfume |
| 10.67 | g | Butane (1.5 bar) |
| 33.33 | g | Propane/Butane |
| Ad 100 | g | Ethanol |

### Example 22

### Pumpspray

| | | |
|---|---|---|
| 2 | g | Polymer P6 |
| 1 | g | Octylacrylamide/Acrylic |
| | | acid/Butylaminoethylmethacrylate/Methacrylate/ |
| | | Hydroxypropylmethacrylate Copolymer |
| 0.3 | g | Perfume |
| 0.1 | g | PEG-12 Dimethicone |
| 10 | g | Water |
| Ad 100 | g | Ethanol |

### Example 23

### Aerosol Coloring Foam

| | | |
|---|---|---|
| 0.5 | g | Polymer P7 |
| 1.00 | g | Polyquaternium-11 (Luviquat ® PQ 11) |
| 0.11 | g | 3-(((2-Nitro-4-(trifluormethyl)phenyl)amino)- |
| | | 1,2-propandiol |
| 0.20 | g | 1,2-Propyleneglycol |
| 0.17 | g | Perfume |
| 0.10 | g | Cetyltrimethylammoniumchloride |
| 6.00 | g | Propane/Butane (5.0 bar) |
| 18.66 | g | Ethanol |
| Ad 100 | g | Water |

### Example 24

### Coloring Hair Foam Fixative

| | | |
|---|---|---|
| 1 | g | Polymer P8 |
| 1.00 | g | Polyquaternium-11 (Gafquat ® 755) |
| 20.10 | g | Colorona Carmine Red |
| 0.20 | g | 1,2-Propyleneglycol |
| 0.17 | g | Perfume |
| 0.10 | g | Cetyltrimethylammoniumchloride |
| 6.00 | g | Propane/Butane (5.0 bar) |
| 18.66 | g | Ethanol |
| Ad 100 | g | Water |

### Example 25

### Pump-Foam Fixative

| | 3. A | | 4. B | | C | |
|---|---|---|---|---|---|---|
| Polymer P1 | 1.5 | g | | | | |
| Polymer P2 | | | 1.5 | g | | |
| Polymer P3 | | | | | 1.5 | g |
| Polyvinylpyrrolidone (K80) | — | | — | | 1.4 | g |
| Vinylcaprolactame/PVP/ | 1.4 | g | 0.9 | g | — | |
| DMAPA Copolymer | | | | | | |
| Cocamidopropyl | 0.6 | g | 0.6 | g | 0.6 | g |
| Hydroxysultaine | | | | | | |
| Cetrimoniumchloride | 0.25 | g | 0.25 | g | 0.25 | g |
| Betaine | 0.1 | g | 0.1 | g | 0.1 | g |
| Citric acid | 0.1 | g | 0.1 | g | 0.1 | g |
| Perfume | 0.15 | g | 0.15 | g | 0.15 | g |
| Ethanol | 2.5 | g | 2.5 | g | 2.5 | g |
| Water | Ad 100 | g | Ad 100 | g | Ad 100 | g |

### Example 26

### Aerosol-Foam

| | | |
|---|---|---|
| 2 | g | Polymer P4 |
| 0.6 | g | Cyclomethicone |
| 0.5 | g | Propyleneglycol |
| 0.5 | g | Quaternium-80; diquaternary |
| | | Dimethylsiloxane |
| 0.2 | g | Cetyltrimethylammoniumchloride |
| 0.2 | g | Polyquaternium-11 |
| 0.1 | g | PEG-12 Dimethicone |
| 6 | g | Propane/Butane |
| Ad 100 | g | Water |

The shape memory polymer used in examples 17 to 26 may be, partially of in total, be replaced by one of the other polymers P1 to P8, with similar results.

1. A method for the treatment of hair, comprising
applying a composition onto hair, wherein the composition comprises at least one first active principle or first active complex, selected among or formed from compounds, which are, alone or in combination with further compounds, capable, after application to hair and after the carrying out of the treatment of hair described in the following, to provide a shape memory effect, and wherein the composition comprises at least one second active principle, selected among cationic agents;
previously, at the same time or subsequent to the application of the composition, bringing the hair into a desired shape (permanent memory shape) and
fixing the memory shape subsequently by inducing a chemical or physical change of the applied agents;
wherein, after a desired or undesired deformation of the memory shape, the initial memory shape can be recalled by means of a physical stimulation.

2. A method according to claim 1, characterized in that the composition comprises at least two active principles, which alone do not show or only show weak shape memory properties and which, after the combined application to hair in accordance with the method according to claim 1, provide hair with a synergistically increased shape memory effect.

3. A method according to claim 1, wherein
the first active principle is a crosslinkable macromer, which forms after crosslinking a shape memory polymer, wherein the macromer comprises
a) crosslinkable segments, which are crosslinkable by means of a chemical reaction; and
b) thermoplastic segments, which are not chemically crosslinkable;
wherein the memory shape is fixed by means of the chemical crosslinking of the macromer and the therewith associated forming of the shape memory polymer, and
wherein the shape memory polymer possesses at least one transition temperature Tₜᵣₐₙₛ.

4. A method for hair treatment, wherein
a programmed hairdo (permanent shape) obtained in accordance with the method of claim 3, is warmed to a temperature above Tₜᵣₐₙₛ,
wherein the hair is then brought into a second (temporary) shape and wherein the second shape is fixed by means of cooling to a temperature below Tₜᵣₐₙₛ.

5. A method according to claim 3, characterized in that the crosslinkable macromer is selected among compounds of the general formula
A1-(X)ₙ-A2
wherein A1 and A2 designate reactive, chemically crosslinkable groups and wherein —(X)ₙ— designates a divalent, thermoplastic polymer or oligomer segment.

6. A method according to claim 5, characterized in that the crosslinkable macromer is selected among polyesters, oligoesters, polyalkylene glycols, oligoalkylene glycols, polyalkylene carbonates and oligoalkylene carbonates substituted with at least two acrylate groups or methacylate groups.

7. A method according to claim 6, characterized in that the crosslinkable macromer is selected among poly(ε-caprolactone)-dimethacrylate, poly(DL-lactide)-methacrylate, poly(L-lactide-co-glycolide)-dimethacrylate, poly(ethyleneglycol) dimethacrylate, poly(propylenglycol) dimethacrylate, PEG-block-PPG-block-PEG-dimethacrylate, poly(ethylenadipate)-dimethacrylate, hexamethylencarbonate-dimethacrylate.

8. A method in accordance with claim 1, characterized in that the composition comprises additionally one macromer having only one terminal or side chain chemically reactive group.

9. A method according to claim 1, wherein
the first active principle is a shape memory polymer, which comprises
a) at least one hard segment which can be crosslinked by means of physical interactions, having a first transition temperature T′ₜᵣₐₙₛ, which lies above room temperature, and
b) at least one soft segment having a second transition temperature Tₜᵣₐₙₛ which lies below T′ₜᵣₐₙₛ, and
wherein the memory shape is fixed by means of a physical crosslinking of the shape memory polymers.

10. A method according to claim 9, wherein the shaping of the hairs occurs under warming to a temperature of at least T′ₜᵣₐₙₛ and wherein the subsequent fixation of the hairdo occurs by means of cooling to a temperature below T′ₜᵣₐₙₛ.

11. A method for hair treatment, wherein
a programmed hairdo (permanent shape) obtained in accordance with a method according to claim 9, is warmed to a temperature between T′ₜᵣₐₙₛ and Tₜᵣₐₙₛ;
wherein the hair is then brought into a second (temporary) shape and
wherein the second shape is fixed by means of cooling to a temperature below Tₜᵣₐₙₛ.

12. A method for reprogramming of a hairdo (permanent shape) obtained in accordance with the method according to claim 9 into a new permanent shape, wherein
the is hairdo is warmed to a temperature above T′ₜᵣₐₙₛ,
followed by bringing the hair into a new shape, and
followed by fixing the new shape by means of cooling to a temperature below T′ₜᵣₐₙₛ.

13. A method in accordance with claim 9, characterized in that the shape memory polymer possesses a degree of crystallinity of from 3 to 80% and wherein the ratio of the moduli of elasticity below and above Tₜᵣₐₙₛ is at least 20.

14. A method according to claim 9, characterized in that the shape memory polymer is a copolyester urethane.

15. A method according to claim 14, characterized in that the shape memory polymer is the reaction product of (a) two different macrodiols, selected among α, ω-dihydroxypolyesters, α,ω-dihydroxyoligoesters, α,ω-dihydroxypolylactones and α,ω-dihydroxyoligolactones, and (b) at least one diisocyanate.

16. A method for the recovery of a programmed hairdo (permanent shape) obtained by the method according to claim 1, wherein the hairdo in a temporary shape according to claim 4 or in the shape of a hairdo obtained by cold forming, is warmed to a temperature above Tₜᵣₐₙₛ.

17. A method according to claim 1, characterized in that the cationic, second active principle is selected among surfactants having cationic groups or groups which can be rendered cationic, polymers having cationic groups or groups which may be rendered cationic, silicone compounds having cationic groups or groups which can be rendered cationic, cationic protein derivatives, cationic protein hydrolysates and betain.

18. A method according to claim 17, characterized in that the cationic surfactant is selected among compounds of the general formula
N⁽⁺⁾R¹R²R³R⁴ X⁽⁻⁾
wherein R¹ to R⁴, independently, designate aliphatic groups, aromatic groups, alkoxy groups, polyoxy alkylene groups, alkylamido groups, hydroxyalkyl groups, alkaryl groups or aryl groups having 1 to 22 carbons atoms, wherein the alkyl groups may be linear, branched or cyclic and wherein at least one of the groups possesses at least 8 carbon atoms and wherein X⁻ represents an anion.

19. A method according to claim 17, characterized in that the cationic polymer is selected among methylvinylimidazoliumchloride/vinlypyrrolidon-copolymers, quarternized vinylpyrrolidon/dimethylaminoethylmethacrylate-copolymers, dimethyldiallylammoniumchloride/sodiumacrylate/acrylamide-copolymers, vinylpyrrolidon/dimethylaminoethylmethacrylate/vinylcaprolactam-copolymers, vinylpyrrolidon/methacrylamidopropyltrimethylammoniumchloride-copolymers, cationic polysaccharide derivatives, chitosan, chitosan salts, chitosan derivatives.

20. A method in accordance with claim 1, characterized in that the composition comprises the first active principle in an amount from 0.01 to 25 wt % and the second active principle in an amount of from 0.01 to 25 wt %.

21. A hair cosmetic composition, comprising in a suitable cosmetic medium an active complex, wherein the active complex comprises at least one first active principle, selected among compounds which are, alone or in combination with further compounds, capable to, after application to hair and after carrying out a method according to claim 1, to provide the hair with a shape memory effect and wherein the active complex comprises at least one second active principle, selected among cationic agents.

22. A composition according to claim 21, characterized in that the first active principle is either
a macromer which can be crosslinked to a shape memory polymer, wherein the crosslinked shape memory polymer possesses at least one transition temperature Tₜᵣₐₙₛ, and wherein the macromer comprises
a) crosslinkable segments, which are crosslinkable by means of chemical bonds, and
b) thermoplastic segments, which are not chemically crosslinkable; and/or a shape memory polymer, which comprises
A) at least one hard segment which may be crosslinked by means of physical interactions, having a first transition temperature T′ₜᵣₐₙₛ, which lies above room temperature, and
B) at least one soft segment having a second transition temperature Tₜᵣₐₙₛ, which lies below T′ₜᵣₐₙₛ;
and wherein the second active principle is selected among surfactants having cationic groups or groups which may be rendered cationic, polymers having cationic groups or groups which may be rendered cationic, silicone compounds having cationic groups or groups which may be rendered cationic, cationic protein derivatives, cationic proteinhydrolysate derivatives and betain.

23. A composition according to claim 22, characterized in that the cationic surfactant is selected among compounds of the general formula
N⁽⁺⁾R¹R²R³R⁴ X⁽⁻⁾
wherein R¹ to R⁴, independently, designate aliphatic groups, aromatic groups, alkoxy groups, polyoxy alkylene groups, alkylamido groups, hydroxyalkyl groups, alkaryl groups or aryl groups having 1 to 22 carbons atoms, wherein the alkyl groups may be linear, branched or cyclic and wherein at least one of the groups possesses at least 8 carbon atoms and wherein X⁻ represents an anion.

24. A composition according to claim 22, characterized in that the cationic polymer is selected among methylvinylimidazoliumchloride/vinlypyrrolidon-copolymers, quarternized vinylpyrrolidon/dimethylaminoethylmethacrylate-copolymers, dimethyldiallylammoniumchloride/sodiumacrylate/acrylamide-copolymers, vinylpyrrolidon/dimethylaminoethylmethacrylate/vinylcaprolactam-copolymers, vinylpyrrolidon/methacrylamidopropyltrimethylammoniumchloride-copolymers, cationic polysaccharide derivatives, chitosan, chitosan salts, chitosan derivatives.

25. A composition according to claim 22, characterized in that the shape memory polymer is contained in an amount of from 0.01 to 25 wt % and wherein the cationic agent is contained in an amount of from 0.01 to 25 wt %.

26. A composition according to claim 21, characterized in that at least two active principles are contained, which show alone no or only weak shape memory properties but which, after combined application onto hair according to claim 1, provide a synergistically increased shape memory effect.

27. A cosmetic preparation, comprising a composition according to claim 21, characterized in that the preparation is present in the form of a lotion, a spray lotion, a cream, a gel, a gelfoam and aerolsprays, a non-aerosolspray, and aerosolfoam, a non-aerosolfoam, a O/W- or W/O emulsion, a micro emulsion or a hair wax.

28. A preparation according to claim 27, characterized in that additionally 0.01 to 25 wt % of at least one active principle are contained, selected among hair conditioning agents, hair fixative agents and hair coloring agents.

29. A method for the recovery of a programmed hairdo (permanent shape) obtained by the method according to claim 1, wherein the hairdo in a temporary shape according to claim 11 or in the shape of a hairdo obtained by cold forming, is warmed to a temperature above Tₜᵣₐₙₛ.
